(19) United States
(12) Patent Application Publication
(10) Pub. No.: US 2021/0174147 A1
(43) Pub. Date: Jun. 10, 2021

(54) OPERATING METHOD OF IMAGE PROCESSING APPARATUS, IMAGE PROCESSING APPARATUS, AND COMPUTER-READABLE RECORDING MEDIUM

(71) Applicant: OLYMPUS CORPORATION, Tokyo (JP)

(72) Inventor: Takeshi OTSUKA, Tokyo (JP)

(73) Assignee: OLYMPUS CORPORATION, Tokyo (JP)

(21) Appl. No.: 17/182,643

(22) Filed: Feb. 23, 2021

Related U.S. Application Data
(63) Continuation of application No. PCT/JP2018/037633, filed on Oct. 9, 2018.

(51) Int. Cl. G06K 9/62; G06K 9/46; G06T 11/00; G06K 9/00; G06N 20/00; G06N 5/04; G16H 10/40; G16H 30/40

(52) U.S. Cl. CPC G06K 9/6257; G06K 9/4652; G06T 11/001; G06K 9/628; G06K 9/00147; G06T 2210/41; G06N 5/04; G16H 10/40; G16H 30/40; G06K 2209/05; G06N 20/00

(57) ABSTRACT

An operating method of an image processing apparatus includes: estimating, from an optical spectrum of each pixel of each training image of training images prepared by first specimen preparing process protocols, staining characteristics of each pigment at the pixel; recording the first specimen preparing process protocol for the training image in association with the estimated staining characteristics; estimating, from an optical spectrum of each pixel of an input training image, staining characteristics of each pigment at the pixel of the input training image prepared by a second specimen preparing process protocol; converting the staining characteristics of at least one selected pigment in the input training image into the staining characteristics of the at least one selected pigment of any one of the training images; and generating, based on the converted staining characteristics, a virtual stained specimen image that is stained by a third specimen preparing process protocol.

### CROSS-REFERENCE TO RELATED APPLICATION

This application is a continuation of International Application No. PCT/JP2018/037633, filed on Oct. 9, 2018, the entire contents of which are incorporated herein by reference.

### BACKGROUND

### 1. Technical Field

The present disclosure relates to an operating method of an image processing apparatus, the image processing apparatus, and a computer-readable recording medium.

### 2. Related Art

Pathological diagnosis involves preparing pathological specimens by processing including cutting, fixation, embedding, sectioning, staining, and mounting, of specimens excised from patients. A pathological specimen is observed using a microscope and the presence or absence of a disease and the extent of the disease are diagnosed from the tissue form and the stained state of the pathological specimen.

In a pathological diagnosis, a primary diagnosis is made, and if a disease is suspected, a secondary diagnosis is made. In the primary diagnosis, the presence or absence of a disease is diagnosed from the tissue form of a pathological specimen. For example, a specimen is subjected to hematoxylin-eosin staining (HE staining) so that the cell nuclei and bone tissues, for example, are stained violet-blue and cytoplasm, connective tissues, and erythrocytes, for example, are stained red. A pathologist morphologically diagnoses the presence or absence of a disease, based on the tissue form.

In the secondary diagnosis, the presence or absence of the disease is diagnosed from expression of molecules. For example, a specimen is subjected to immunostaining to visualize expression of molecules from antigen-antibody reactions. A pathologist then diagnoses the presence or absence of the disease from the expression of molecules. The pathologist also selects an appropriate treatment method from the positive rate (the ratio between the negative cells and the positive cells).

Images of a pathological specimen may be formed by connecting a camera to a microscope and imaging the pathological specimen. Images of the whole pathological specimen may be formed by a virtual microscope system (a virtual slide system). Formation of images of a pathological specimen enables utilization of the images for education and telepathology, for example.

Methods for supporting diagnosis by image processing of pathological specimen images have also been developed. Methods for supporting diagnosis include: a method in which a pathologist's diagnosis is imitated by image processing; and a method in which machine learning is performed using a large number of training images. Linear discrimination and deep learning are used for the machine learning, for example. The burden placed on a pathologist in pathological diagnosis is getting larger due to the shortage of pathologists, for example. Therefore, there is a demand for diagnostic support to reduce the burdens on pathologists.

Deep learning has a function of being able to automatically compute preset feature data, is starting to be put to practical use with the advancement of computational resources, and has been utilized for a wide range of applications, mainly for image recognition and voice recognition. Deep learning is used in analysis of pathological specimen images and “Accurate and reproducible invasive breast cancer detection in whole-slide images: A Deep Learning approach for quantifying tumor extent”, Scientific Reports, 2017 Apr. 18; 7:46450, doi: 10.1038/srep46450, discloses a method of highly accurately detecting breast cancer from pathological specimen images, using deep learning. Deep learning readily enables highly accurate diagnostic support as compared to conventional image processing, if a large number of accurate training images are able to be prepared.

Pathological specimen images vary in color due to various reasons. For example, specimens prepared vary in color, for example, in staining concentration, depending on the preferences of the pathologists, the skill of the clinical technologists, and the performance of the specimen preparing equipment. Therefore, if discrepancy in color of a pathological specimen image is outside the range of the training images, diagnostic support is unable to be performed appropriately for that image. Accordingly, a large number of training images generated by different specimen preparing processes at plural specimen preparing institutions needs to be collected to perform appropriate diagnostic support.

### SUMMARY

In some embodiments, an operating method of an image processing apparatus includes: estimating, from an optical spectrum of each pixel of each training image of plural training images, a pigment spectrum and a pigment quantity that are staining characteristics of each pigment at the pixel of the training image, the plural training images being stained specimen images prepared by plural first specimen preparing process protocols different from one another, each first specimen preparing process protocol including staining using plural pigments; recording the first specimen preparing process protocol for the training image in association with the estimated staining characteristics; estimating, from an optical spectrum of each pixel of an input training image, staining characteristics of each pigment at the pixel of the input training image, the input training image being a stained specimen image prepared by a second specimen preparing process protocol different from the plural first specimen preparing process protocols and including staining using the plural pigments, the input training image being input as a training image for learning; converting the staining characteristics of at least one selected pigment in the input training image into the staining characteristics of the at least one selected pigment of any one of the plural training images; and generating, based on the converted staining characteristics of the at least one selected pigment in the input training image, a virtual stained specimen image that is stained by a third specimen preparing process protocol different from the first specimen preparing process protocols and the second specimen preparing process protocol.

In some embodiments, an image processing apparatus includes a processor including hardware. The processor is configured to: estimate, from an optical spectrum of each pixel of each training image of plural training images, a pigment spectrum and a pigment quantity that are staining characteristics of each pigment at the pixel of the training image, the plural training images being stained specimen images prepared by plural first specimen preparing process protocols different from one another, each first specimen preparing process protocol including staining using plural pigments; record the first specimen preparing process protocol for the training image in association with the estimated staining characteristics; estimate, from an optical spectrum of each pixel of an input training image, staining characteristics of each pigment at the pixel of the input training image, the input training image being a stained specimen image prepared by a second specimen preparing process protocol different from the plural first specimen preparing process protocols and including staining using the plural pigments, the input training image being input as a training image for learning; convert the staining characteristics of at least one selected pigment in the input training image into the staining characteristics of the at least one selected pigment of any one of the plural training images; and generate, based on the converted staining characteristics of the at least one selected pigment in the input training image, a virtual stained specimen image that is stained by a third specimen preparing process protocol different from the first specimen preparing process protocols and the second specimen preparing process protocol.

In some embodiments, provided is a non-transitory computer-readable recording medium with an executable program stored thereon. The program causes an image processing apparatus to execute: estimating, from an optical spectrum of each pixel of each training image of plural training images, a pigment spectrum and a pigment quantity that are staining characteristics of each pigment at the pixel of the training image, the plural training images being stained specimen images prepared by plural first specimen preparing process protocols different from one another, each first specimen preparing process protocol including staining using plural pigments; recording the first specimen preparing process protocol for the training image in association with the estimated staining characteristics; estimating, from an optical spectrum of each pixel of an input training image, staining characteristics of each pigment at the pixel of the input training image, the input training image being a stained specimen image prepared by a second specimen preparing process protocol different from the plural first specimen preparing process protocols and including staining using the plural pigments, the input training image being input as a training image for learning; converting the staining characteristics of at least one selected pigment in the input training image into the staining characteristics of the at least one selected pigment of any one of the plural training images; and generating, based on the converted staining characteristics of the at least one selected pigment in the input training image, a virtual stained specimen image that is stained by a third specimen preparing process protocol different from the first specimen preparing process protocols and the second specimen preparing process protocol.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating an example of a configuration of an imaging system including an image processing apparatus according to a first embodiment of the disclosure;

FIG. 2 is a flowchart illustrating operation of the image processing apparatus illustrated in FIG. 1;

FIG. 3 is a diagram illustrating a table of specimen preparing process protocols;

FIG. 4 is a diagram illustrating an optical spectrum of one pixel in an input training image;

FIG. 5 is a diagram illustrating an H pigment spectrum of the one pixel in the input training image;

FIG. 6 is a diagram illustrating an H pigment quantity of the one pixel in the input training image;

FIG. 7 is a diagram illustrating a DAB pigment spectrum of the one pixel in the input training image;

FIG. 8 is a diagram illustrating a DAB pigment quantity of the one pixel in the input training image;

FIG. 9 is a diagram illustrating an H pigment spectrum of a training image for conversion;

FIG. 10 is a diagram illustrating an H pigment quantity of the training image for conversion;

FIG. 11 is a block diagram illustrating an example of a configuration of an imaging system including an image processing apparatus according to a second embodiment of the disclosure;

FIG. 12 is a flowchart illustrating operation of the image processing apparatus illustrated in FIG. 11;

FIG. 13 is a diagram illustrating a table of specimen preparing process protocols;

FIG. 14 is a diagram illustrating H pigment spectra at pixels in an input training image;

FIG. 15 is a diagram illustrating DAB pigment spectra at the pixels in the input training image;

FIG. 16 is a diagram illustrating how a cell nucleus and cytoplasm are separated;

FIG. 17 is a diagram illustrating H pigment spectra of a cell nucleus and cytoplasm;

FIG. 18 is a diagram illustrating H pigment quantities in the cell nucleus and cytoplasm;

FIG. 19 is a diagram illustrating DAB pigment spectra of the cell nucleus and cytoplasm;

FIG. 20 is a diagram illustrating DAB pigment quantities in the cell nucleus and cytoplasm;

FIG. 21 is a diagram illustrating H pigment spectra of a cell nucleus and cytoplasm in a training image for conversion;

FIG. 22 is a diagram illustrating H pigment quantities in the cell nucleus and cytoplasm in the training image for conversion;

FIG. 23 is a block diagram illustrating an example of a configuration of an imaging system including an image processing apparatus according to a third embodiment of the disclosure;

FIG. 24 is a flowchart illustrating operation of the image processing apparatus illustrated in FIG. 23; and

FIG. 25 is a block diagram illustrating an example of a configuration of an imaging system including an image processing apparatus according to a fourth embodiment of the disclosure.

### DETAILED DESCRIPTION

An operating method of an image processing apparatus, the image processing apparatus, and an operating program for the image processing apparatus, according to the disclosure will be described below by reference to the drawings. The disclosure is not limited by these embodiments. The disclosure is generally applicable to an operating method of an image processing apparatus, the image processing apparatus, and an operating program for the image processing apparatus that are for supporting diagnosis using plural training images.

Any elements that are the same or corresponding to each other are assigned with the same reference sign throughout the drawings, as appropriate. It also needs to be noted that the drawings are schematic and relations between dimensions of each element therein and proportions between the elements therein may be different from the actual ones. The drawings may also include a portion that differs in its dimensional relations or proportions between the drawings.

### First Embodiment

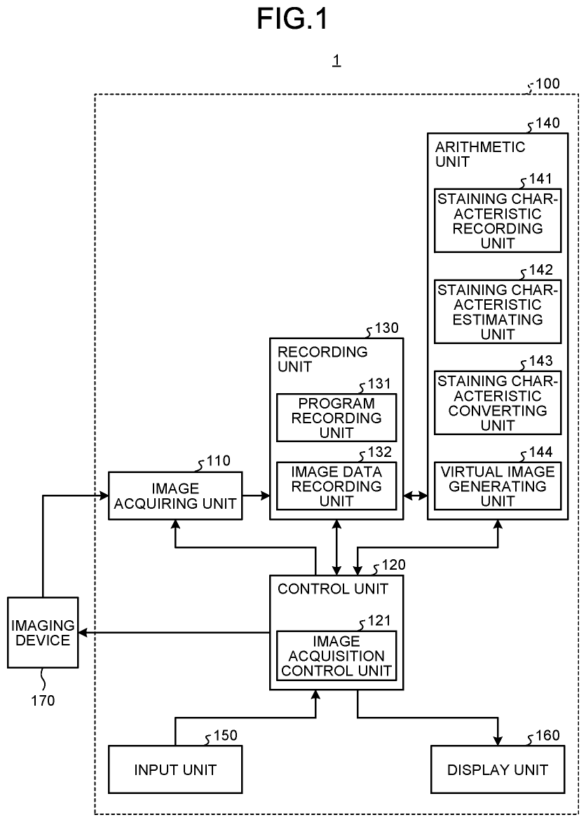

FIG. 1 is a block diagram illustrating an example of a configuration of an imaging system including an image processing apparatus according to a first embodiment of the disclosure. As illustrated in FIG. 1, an imaging system 1 includes: an imaging device 170, such as a fluorescence microscope; and an image processing apparatus 100 formed of a computer, such as a personal computer, that is connectable to the imaging device 170.

The image processing apparatus 100 includes: an image acquiring unit 110 that acquires image data from the imaging device 170; a control unit 120 that controls the overall operation of the system including the image processing apparatus 100 and the imaging device 170; a recording unit 130 that stores therein, for example, image data acquired by the image acquiring unit 110; an arithmetic unit 140 that executes predetermined image processing, based on the image data stored in the recording unit 130; an input unit 150; and a display unit 160.

The image acquiring unit 110 is configured, as appropriate, according to the form of the system including the image processing apparatus 100. For example, when the imaging device 170 is connected to the image processing apparatus 100, the image acquiring unit 110 includes an interface that fetches image data output from the imaging device 170. Furthermore, if a server to save image data generated by the imaging device 170 is installed, the image acquiring unit 110 includes, for example, a communication device connected to the server and performs data communication with the server to acquire image data. Or, the image acquiring unit 110 may include a reader device to which a portable recording medium is detachably attached and which reads image data recorded in the recording medium.

The control unit 120 is formed using a general-purpose processor, such as a central processing unit (CPU), or a special-purpose processor, such as an arithmetic circuit that executes a specific function, like an application specific integrated circuit (ASIC). If the control unit 120 is a general-purpose processor, for example, the control unit 120 transfers instructions and data to the respective units forming the image processing apparatus 100 to integrally control the overall operation of the image processing apparatus 100, by reading various programs stored in the recording unit 130. If the control unit 120 is a special-purpose processor: the processor may execute various kinds of processing alone; or the processor and the recording unit 130 may cooperate or be united with each other to execute various kinds of processing, by using, for example, various data stored in the recording unit 130.

The control unit 120 has an image acquisition control unit 121 that controls operation of the image acquiring unit 110 and imaging device 170 to acquire an image, and controls the operation of the image acquiring unit 110 and imaging device 170, based on an input signal input from the input unit 150, an image input from the image acquiring unit 110, and, for example, a program and data stored in the recording unit 130.

The recording unit 130 includes: various IC memories including a read only memory (ROM) and a random access memory (RAM), like rewritable flash memories; an information storage device, such as a built-in hard disk, a hard disk that is connected via a data communication terminal, or a DVD-ROM; and a device that writes and reads information into and from the information storage device. The recording unit 130 includes a program recording unit 131 that stores therein an image processing program, and an image data recording unit 132 that stores therein image data and various parameters that are used during execution of the image processing program.

The arithmetic unit 140 is formed using a general-purpose processor, such as a CPU or a graphics processing unit (GPU), or a special-purpose processor, such as an arithmetic circuit that executes a specific function, like an ASIC. If the arithmetic unit 140 is a general-purpose processor, the arithmetic unit 140 executes image processing for estimating a depth at which a specific tissue is present, based on a multiband image, by reading the image processing program stored in the program recording unit 131. If the arithmetic unit 140 is a special-purpose processor: the processor may execute various kinds of processing alone; or the processor and the recording unit 130 may cooperate or be united with each other to execute image processing, by using, for example, various data stored in the recording unit 130.

More particularly, the arithmetic unit 140 includes a staining characteristic recording unit 141, a staining characteristic estimating unit 142, a staining characteristic converting unit 143, and a virtual image generating unit 144.

The staining characteristic recording unit 141 estimates, from an optical spectrum of each pixel of each training image of plural training images that are stained specimen images prepared by plural specimen preparing process protocols different from each other and including staining using plural pigments, a pigment spectrum and a pigment quantity that are staining characteristics of each pigment at that pixel, and records the specimen preparing process protocol of that training image in association with the estimated staining characteristics, into the recording unit 130.

The staining characteristic estimating unit 142 estimates, from an optical spectrum of each pixel of an input training image that is a stained specimen image that has been prepared by a specimen preparing process protocol different from the specimen preparing process protocols of the plural training images, staining characteristics of each pigment at that pixel, the specimen preparing process protocol including staining using the plural pigments, the input training image being input as a training image for learning.

The staining characteristic converting unit 143 repeatedly converts staining characteristics of each pigment in the input training image, into staining characteristics of that pigment of each training image recorded in the recording unit 130. The staining characteristic converting unit 143 may just convert staining characteristics of at least one selected pigment of the input training image, into staining characteristics of the selected pigment in any one training image of the plural training images.

Based on the pigment's staining characteristics resulting from the conversion by the staining characteristic converting unit 143, the virtual image generating unit 144 generates a virtual stained specimen image stained by a specimen preparing process protocol different from the specimen preparing process protocols for the plural training images and input training image. Specifically, based each pigment's staining characteristics resulting from the conversion by the staining characteristic converting unit 143, the virtual image generating unit 144 repeatedly generates a virtual stained specimen image stained by a specimen preparing process protocol different from the specimen preparing process protocols of the input training image and plural training images.

The input unit 150 is formed of any of various input devices, such as a keyboard and a mouse, a touch panel, and various switches, for example, and outputs an input signal that is in response to input of operation.

The display unit 160 is implemented by a display device, such as a liquid crystal display (LCD), an electroluminescence (EL) display, or a cathode ray tube (CRT) display, and displays various screens thereon, based on display signals input from the control unit 120.

The imaging device 170 includes, for example, an imaging element, such as a charge coupled device (CCD) or a complementary metal oxide semiconductor (CMOS), and under control by the image acquiring unit 110 in the image processing apparatus 100, converts light incident on a light receiving surface of the imaging element, into an electric signal corresponding to intensity of the light and outputs the electric signal as image data. The imaging device 170 may include an RGB camera, and capture an RGB image or capture a multiband image. Methods of multiband imaging include a method of changing wavelength of illumination light, a method of providing a filter on an optical path of light from a light source to change wavelength of the light transmitted through the filter, the light being white light, and a method of using a multicolor sensor. Examples of the method of providing a filter on an optical path include a method of using plural bandpass filters that transmit wavelengths different from each other, a method of using a diffraction grating, and a method of using a liquid crystal tunable filter or an acoustic tunable filter. The optical path may be branched and the branched light may be simultaneously received by plural cameras having different spectral characteristics.

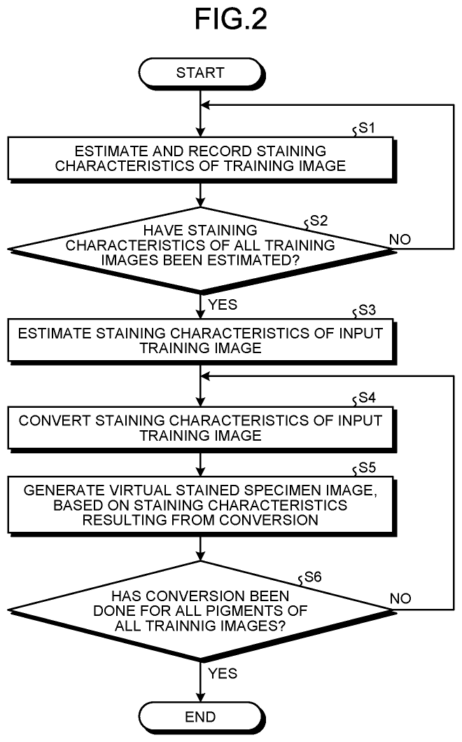

Next, processing for generating a virtual training image using the imaging system according to the first embodiment will be described below. FIG. 2 is a flowchart illustrating operation of the image processing apparatus illustrated in FIG. 1. As illustrated in FIG. 2, firstly, the staining characteristic recording unit 141 estimates, from an optical spectrum of each pixel of a training image, a pigment spectrum and a pigment quantity that are staining characteristics of each pigment at that pixel, and records the specimen preparing process protocol for that training image in association with the estimated staining characteristics, into the recording unit 130 (Step S1).

Subsequently, the arithmetic unit 140 determines whether or not staining characteristics for all training images have been estimated (Step S2). If the arithmetic unit 140 determines that staining characteristics for all of the training images have not been estimated, the processing is returned to Step S1 and the same processing is repeated. That is, the arithmetic unit 140 repeats the same processing until staining characteristics of all of the training images have been estimated.

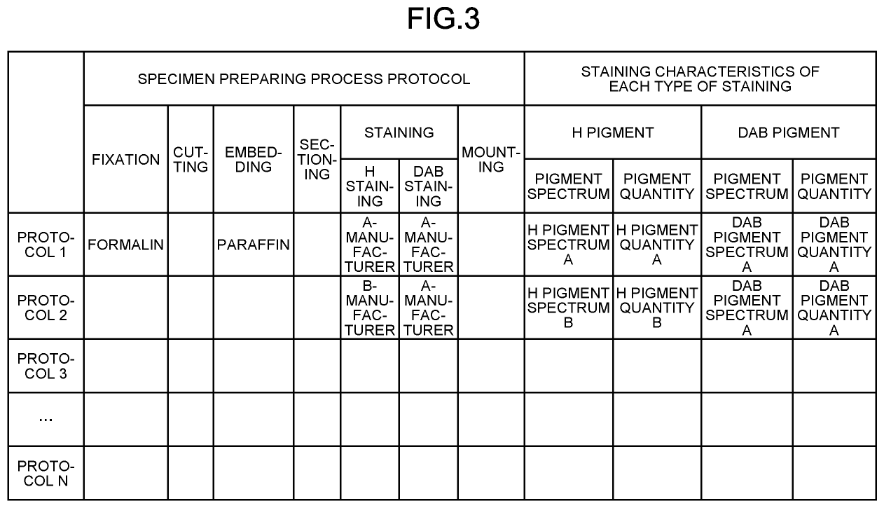

FIG. 3 is a diagram illustrating a table of specimen preparing process protocols. As illustrated in FIG. 3, through the processing at Steps S1 and S2, the staining characteristic recording unit 141: estimates a pigment spectrum and a pigment quantity that are staining characteristics for each training image of N training images that are stained specimen images stained by, for example, hematoxylin (H) staining and diaminobenzidine (DAB) staining and prepared by plural specimen preparing process protocols (protocols 1, 2, . . . , N) that are different from one another; and records the plural specimen preparing process protocols and the estimated staining characteristics in association with each other, into the recording unit 130. As a result, a database of staining characteristics illustrated in FIG. 3 is able to be generated. In the column for the specimen preparing process protocols, in the database illustrated in FIG. 3, processes for preparation of the training images have been recorded. Specifically, conditions for fixation, cutting, embedding, sectioning, staining, and mounting, and types of reagents used, for example, are recorded as specimen preparing process protocols. Furthermore, in the column for staining characteristics in the database illustrated in FIG. 3, pigment spectra and pigment quantities estimated respectively for H pigment and DAB pigment have been stored. Only the names of files having the staining characteristics recorded therein are written in FIG. 3, and for example, a data group for H pigment spectra at pixels of training images have been recorded in the file, “H pigment spectrum A”, and a data group for H pigment quantities at the pixels of the training images have been recorded in the file, “H pigment quantity A”. In this first embodiment, the example in which staining is performed by hematoxylin staining and DAB staining is described, but staining may include any other counterstaining, special staining, or immunostaining. Thereafter, the staining characteristic estimating unit 142 estimates, from an optical spectrum of each pixel of an input training image, staining characteristics of each pigment at that pixel (Step S3). The input training image is input as a training image for learning and is a stained specimen image prepared by a specimen preparing process protocol including staining by H staining and DAB staining; and that specimen preparing process protocol is a specimen preparing process protocol different from each of those of the N training images.

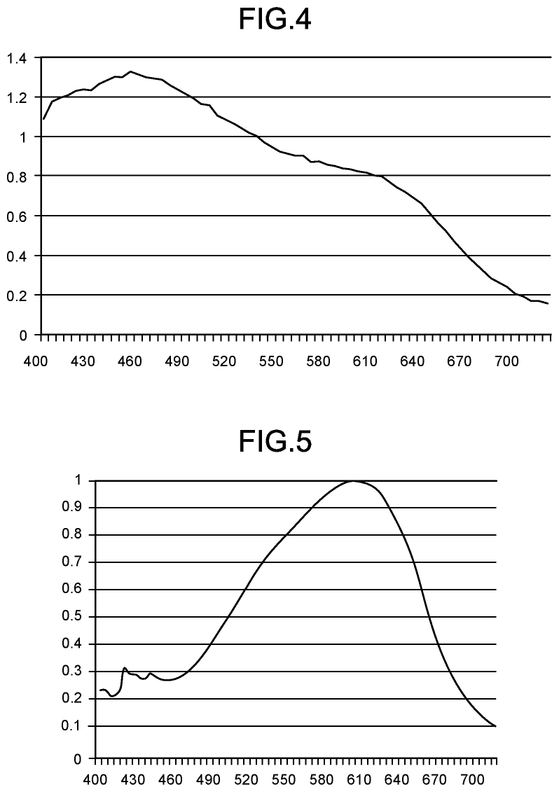

FIG. 4 is a diagram illustrating an optical spectrum of one pixel of an input training image. The staining characteristic estimating unit 142 estimates an optical spectrum like the one illustrated in FIG. 4, for each pixel of the input training image.

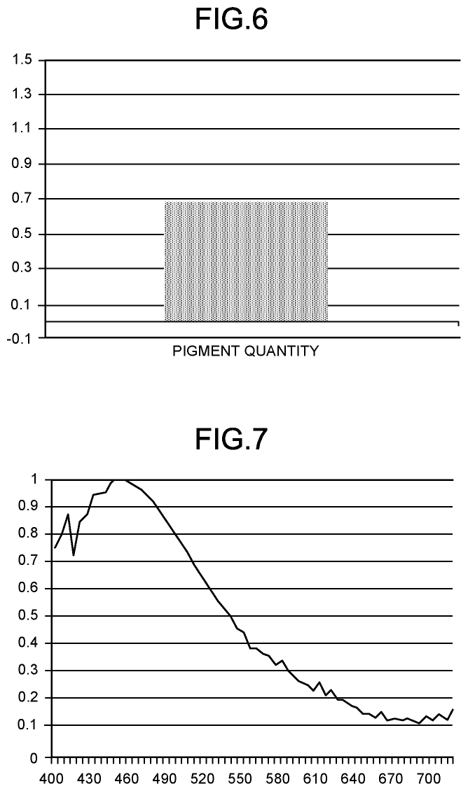

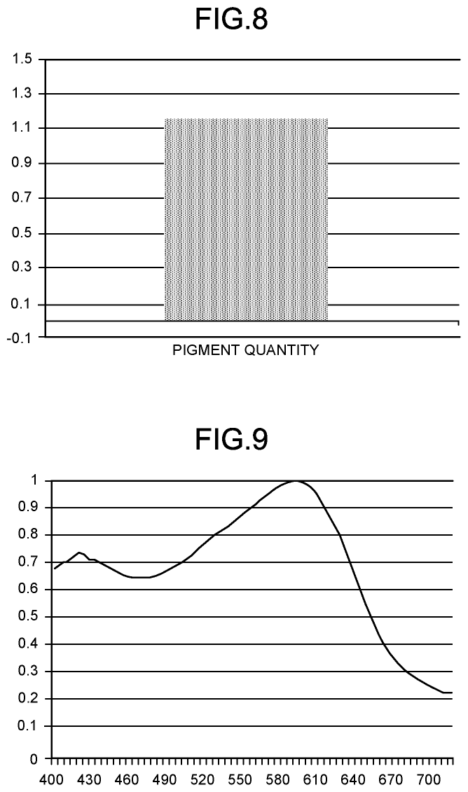

FIG. 5 is a diagram illustrating an H pigment spectrum of the one pixel of the input training image. FIG. 6 is a diagram illustrating an H pigment quantity of the one pixel of the input training image. FIG. 7 is a diagram illustrating a DAB pigment spectrum of the one pixel of the input training image. FIG. 8 is a diagram illustrating a DAB pigment quantity of the one pixel of the input training image. For each pixel in the input training image, the staining characteristic estimating unit 142 estimates, from the optical spectrum illustrated in FIG. 4, each of the H pigment spectrum illustrated in FIG. 5, the H pigment quantity illustrated in FIG. 6, the DAB pigment spectrum illustrated in FIG. 7, and the DAB pigment quantity illustrated in FIG. 8.

Staining characteristics may be estimated from a spectral image or an optical spectrum may be estimated from an input image. Japanese Patent Application Laid-open No. 2009-270890 discloses a method of estimating an optical spectrum from a multiband image. When a pathological specimen is observed using transmitted light, because the specimen is thin and absorption is dominant, a pigment quantity may be estimated based on the Lambert-Beer's law. When a pigment quantity is estimated using a small number of bands, various measures are preferably taken for accurate estimation of the pigment quantity. Japanese Patent Application Laid-open No. 2011-53074, Japanese Patent Application Laid-open No. 2009-8481, and Japanese Patent Application Laid-open No. 2012-207961 disclose methods of estimating pigment spectra from input images. A method, such as a method of using plural pigment spectra, a method of correcting a pigment spectrum to match a measured spectrum, or a method of correcting a pigment spectrum based on a changing model may be selected from these references, as appropriate.

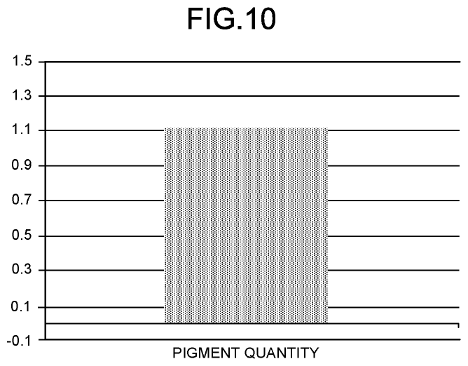

The staining characteristic converting unit 143 converts the staining characteristics of each pigment in the input training image, into staining characteristics of the pigment of a training image recorded in the recording unit 130 (Step S4). FIG. 9 is a diagram illustrating an H pigment spectrum of a training image for conversion. FIG. 10 is a diagram illustrating an H pigment quantity of the training image for conversion. FIG. 9 and FIG. 10 respectively correspond to, for example, the H pigment spectrum A and H pigment quantity A of the protocol 1 illustrated in FIG. 3. The staining characteristic converting unit 143 then converts the H pigment spectrum of the input training image illustrated in FIG. 4 into the H pigment spectrum of the training image illustrated in FIG. 9 and converts the H pigment quantity of the input training image illustrated in FIG. 5 into the H pigment quantity of the training image illustrated in FIG. 10. The H pigment spectrum of each pixel of the input training image may be converted into, for example, a spectrum that is an average of H pigment spectra of that pixel in the training images. Furthermore, the H pigment quantity may be calculated according to, for example, a ratio between the maximum value of the H pigment spectrum of each pixel of the input training image and the maximum value of a spectrum that is an average of H pigment spectra of that pixel in the training images. Similar methods may be used in conversion for the DAB pigment.

Based on the pigments' staining characteristics resulting from the conversion done by the staining characteristic converting unit 143, the virtual image generating unit 144 repeatedly generates a virtual stained specimen image stained by a specimen preparing process protocol different from the specimen preparing process protocols of the input training image and plural training images (Step S5).

Subsequently, the arithmetic unit 140 determines whether or not conversion for all pigments in all training images has been done (Step S6). If the arithmetic unit 140 determines that conversion has not been done for all of the pigments in all of the training images, the processing is returned to Step S4 and the same processing is repeated. That is, the arithmetic unit 140 repeats the processing until conversion has been done for all of the pigments in all of the training images. As a result, conversion of the input training image for the training images prepared by N different specimen preparing process protocols is done. Including both the conversion for the H pigment and the conversion for the DAB pigment, a total of 2N virtual stained specimen images are able to be generated.

A specific calculation method will be described below. Firstly, an absorbance a′(x, y, λ) after conversion expressed using coordinates (x,y) and a wavelength λ may be expressed by Equation (1) below, where a reference pigment quantity of any pigment in any training image is D, a reference spectrum of the pigment in the training image is A(λ), and a pigment quantity of the pigment at the coordinates (x,y) is d(x,y). The subscript H represents the pigment quantity in hematoxylin staining, the subscript DAB represents the pigment quantity in DAB staining, the subscript src represents the pigment quantity before conversion, and the subscript dest represents the pigment quantity after conversion.

a′(x,y,λ)=D_{H·d·dest}/D_{H·n·src}·A_{H·dest}(λ)·d_{H}(x,y)+D_{DAB·dest}/D_{DAB·src}·A_{DAB·dest}(λ)·d_{DAB}(x,y) (1)

Subsequently, a transmittance s(x, y, λ) after conversion may be expressed by Equation (2) below using the absorbance a′(x, y, λ) after conversion found by Equation (1).

s(x,y,λ)=e^{-a′(x,y,λ)} (2)

Furthermore, an sRGB image may be expressed by Equations (3) to (5) below using the transmittance s(x,y,k) after conversion found by Equation (2). In these equations, X(x,y), Y(x,y), and Z(x,y) are values of the coordinates (x,y) in an XYZ color space after conversion. Furthermore, f_{X}(λ), f_{Y}(λ), and f_{Z}(λ) are values of XYZ color functions.

X(x,y)=∫_{Λ}s(x,y,λ)·f_{X}(λ)dλ (3)

Y(x,y)=∫_{Λ}s(x,y,λ)·f_{Y}(λ)dλ (4)

Z(x,y)=∫_{Λ}s(x,y,λ)·f_{Z}(λ)dλ (5)

From Equation (6) below, sRGBₗᵢₙₑₐᵣ is able to be calculated. In Equation (6), Rₗᵢₙₑₐᵣ, Gₗᵢₙₑₐᵣ, and Bₗᵢₙₑₐᵣ are linear sRGB values after conversion.

$$[ \begin{matrix}{R}_{linear} \\ {G}_{linear} \\ {B}_{linear}\end{matrix} ] = [ \begin{matrix}3.2406 & - 1.53 \; 72 & - 0.4986 \\ - 0.9689 & 1.87 \; 58 & 0.04 l 5 \\ 0.0557 & - 0.2040 & 1.05 \; 70\end{matrix} ] [ \begin{matrix}X \\ Y \\ Z\end{matrix} ] \qquad ( 6 )$$

Furthermore, a nonlinear sRCG image is able to be generated: by calculating C_{srgb}(x,y,b)=12.96═Cₗᵢₙₑₐᵣ (x,y,b), if Cₗᵢₙₑₐᵣ (x,y,b)≤0.0031308(x,y,b); and by calculating C_{srgb}(x,y,b)=1.055•Cₗᵢₙₑₐᵣ (x,y,b)^{1/24}−0.055, if Cₗᵢₙₑₐᵣ (x,y,b) >0.0031308. In these equations, Cₗᵢₙₑₐᵣ is any linear sRGB value after conversion and C_{srgb} is any sRGB value after conversion.

A virtual stained specimen image generated may be not necessarily an RGB image, and may be a special optical image, a multiband image, or a spectral image. If a special optical image or a multiband image is generated as a virtual stained specimen image, the virtual stained specimen image is calculated by multiplying an optical spectrum by camera sensitivity characteristics and illumination characteristics. Filter characteristics may be considered in addition to the camera sensitivity characteristics.

As described above, according to the first embodiment, by conversion between staining characteristics of an input training image and staining characteristics of training images, many pathological specimen images prepared by virtual specimen preparing process protocols are able to be prepared. By using the prepared virtual stained specimen images as new training images, the number of training images is able to be increased significantly. Because this conversion involves conversion maintaining pigment information, the number of training images is able to be increased with the accuracy in diagnostic support maintained. In the first embodiment described above, conversion is done for all pigments in all of training images, but a virtual stained specimen image is able to be prepared as long as conversion is performed for one pigment of at least one training image.

### Second Embodiment

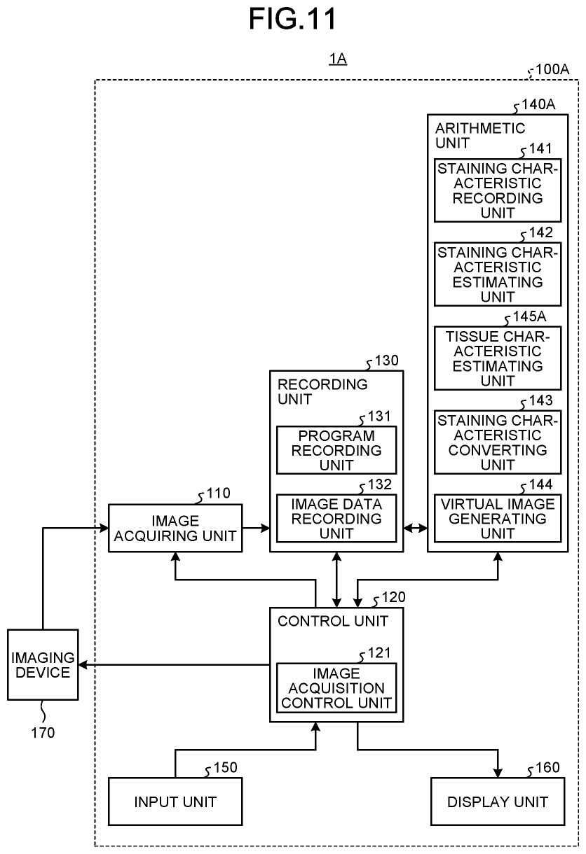

Next, a second embodiment of the disclosure will be described below. FIG. 11 is a block diagram illustrating an example of a configuration of an imaging system including an image processing apparatus according to a second embodiment of the disclosure. As illustrated in FIG. 11, an arithmetic unit 140A of an image processing apparatus 100A in an imaging system 1A includes a tissue characteristic estimating unit 145A that estimates a tissue that each pixel belongs to, from staining characteristics of each pixel in plural training images and an input training image.

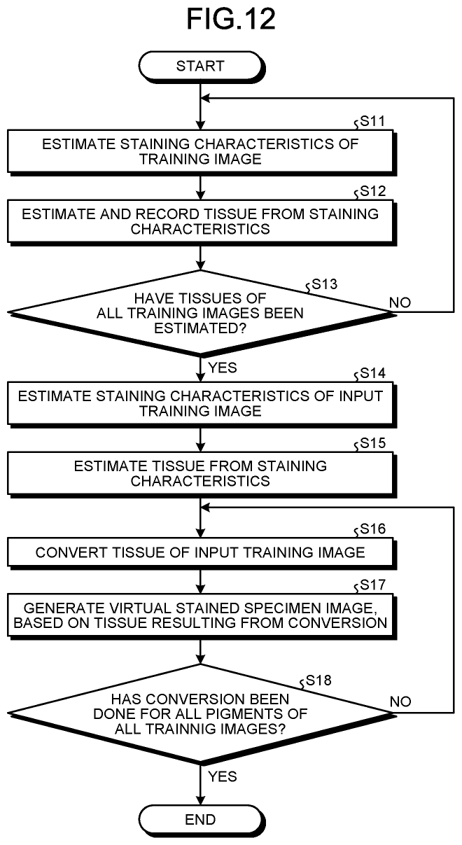

FIG. 12 is a flowchart illustrating operation of the image processing apparatus illustrated in FIG. 11. As illustrated in FIG. 12, similarly to the first embodiment, the staining characteristic recording unit 141 estimates, from an optical spectrum of each pixel in a training image, a pigment spectrum and a pigment quantity that are staining characteristics of each pigment at that pixel (Step S1).

The tissue characteristic estimating unit 145A then estimates a tissue that each pixel in plural training images belongs to, from staining characteristics of that pixel of the plural training images, and records a specimen preparing process protocol for the training image in association with the estimated tissue, into the recording unit 130 (Step S12).

Subsequently, the arithmetic unit 140 determines whether or not tissues have been estimated for all of the training images (Step S13). If the arithmetic unit 140 determines that tissues have not been estimated for all of the training images, the processing is returned to Step S1 and the same is repeated. That is, the arithmetic unit 140 repeats the same processing until tissues have been estimated for all of the training images.

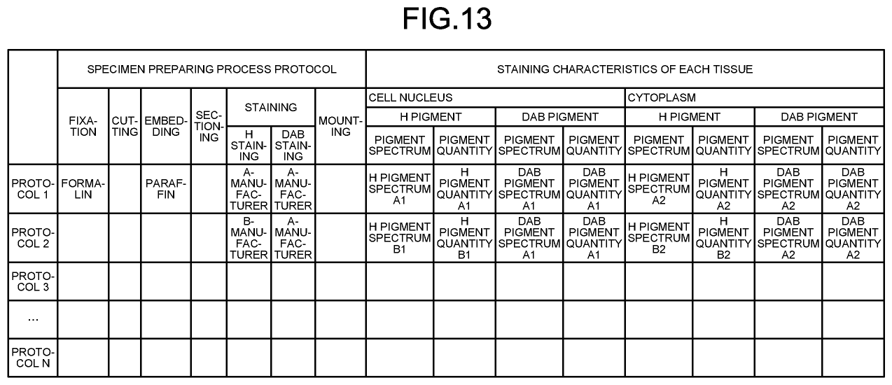

FIG. 13 is a diagram illustrating a table of specimen preparing process protocols. As illustrated in FIG. 13, through the processing at Steps S1 to S13, the arithmetic unit 140 estimates a pigment spectrum and a pigment quantity as staining characteristics of a tissue for each training image of N training images that are stained specimen images stained by, for example, H staining and DAB staining and prepared by plural specimen preparing process protocols (protocols 1, 2, . . . , N) that are different from one another, and records them in association with each other, into the recording unit 130. As a result, a database of the staining characteristics of the tissues illustrated in FIG. 13 is able to be generated. In the column for the specimen preparing process protocols, in the database illustrated in FIG. 13, similarly to FIG. 3 according to the first embodiment, processes performed in preparation of each training image have been recorded. Furthermore, pigment spectra and pigment quantities estimated for each of the H pigment and DAB pigment are stored for each of cell nuclei and cytoplasm, in the column for the staining characteristics of the tissues, in the database illustrated in FIG. 13.

Thereafter, the staining characteristic estimating unit 142 estimates, from an optical spectrum of each pixel in an input training image, staining characteristics of each pigment at that pixel (Step S14).

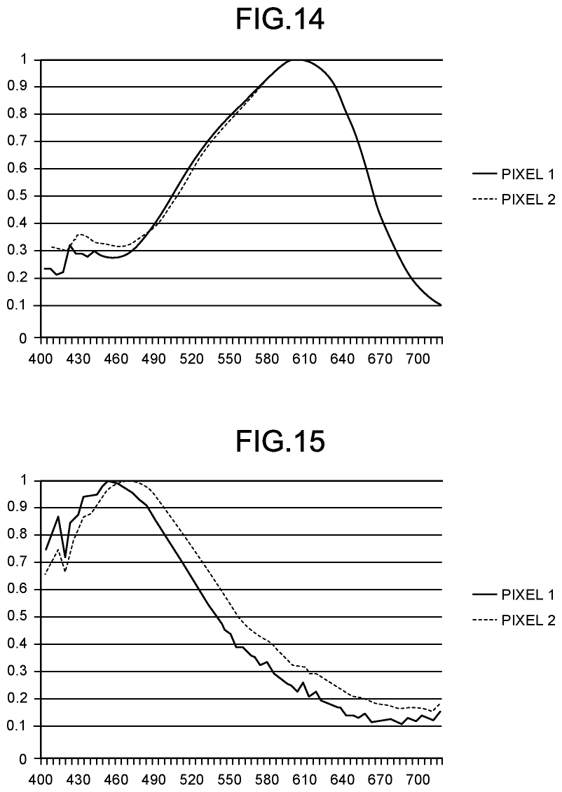

FIG. 14 is a diagram illustrating H pigment spectra at pixels in an input training image. FIG. 15 is a diagram illustrating DAB pigment spectra at the pixels of the input training image. The staining characteristic estimating unit 142 estimates the spectra for these pigments illustrated in FIG. 14 and FIG. 15 from the optical spectrum illustrated in FIG. 4.

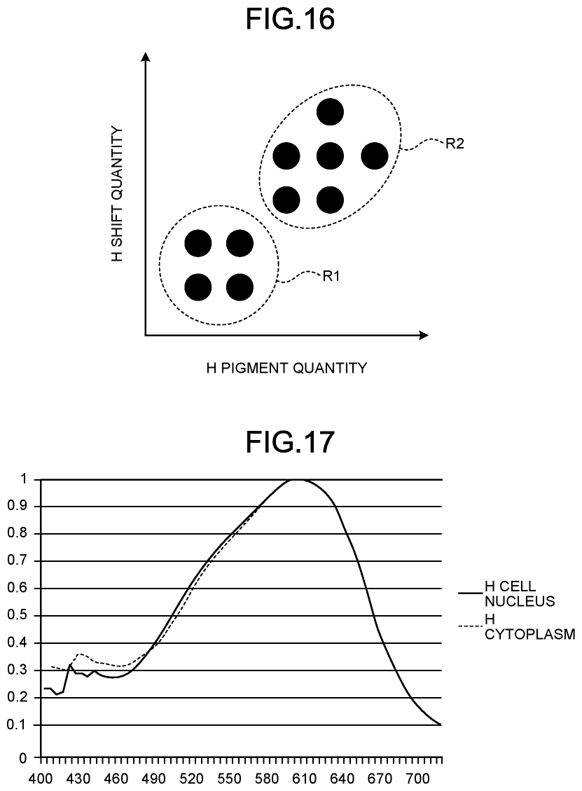

Subsequently, the tissue characteristic estimating unit 145A estimates a tissue that each pixel of the input training image belongs to, from staining characteristics of the pixel (Step S15). FIG. 16 is a diagram illustrating how a cell nucleus and cytoplasm are separated. The tissue characteristic estimating unit 145A estimates and plots an H pigment quantity and an H shift quantity for each pixel from the H pigment spectra illustrated in FIG. 14. According to the region where the plotted point is positioned, each pixel is classified as cytoplasm included in a region R1 or a cell nucleus included in a region R2. An H shift quantity is a value corresponding to a peak of an H pigment spectrum. The tissues are not necessarily cell nuclei nor cytoplasm, and may be any tissues including cell membranes, erythrocytes, fibrae, mucus, and fat.

As disclosed in Japanese Patent Application Laid-open No. 2012-117844 and Japanese Patent Application Laid-open No. 2012-233784, staining characteristics in each tissue may be automatically calculated. As a method of automatically calculating staining characteristics of each tissue, a method may be selected from these references as appropriate, such as a method of classifying tissues from pigment quantity distributions, or a method of classifying tissues from wavelength feature data, such as wavelength shifts. A sample pixel may be manually selected for each tissue to set staining characteristics of the tissue.

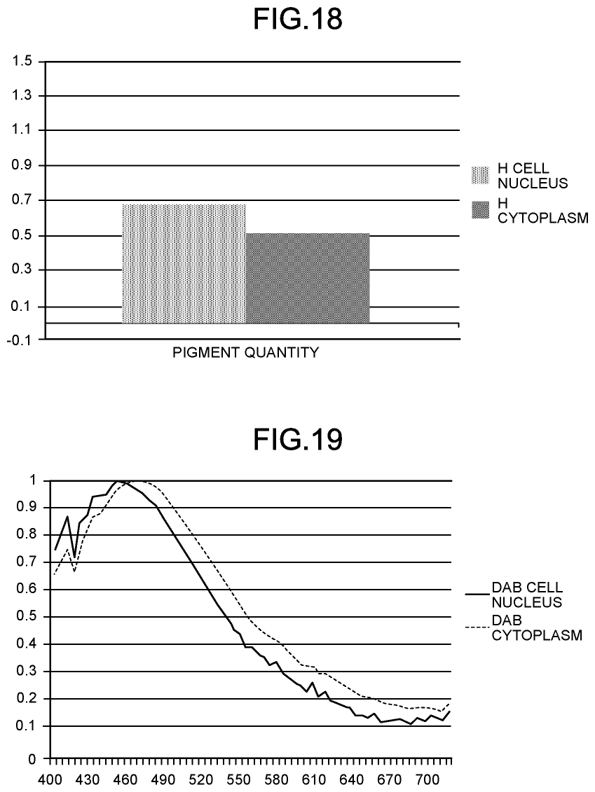

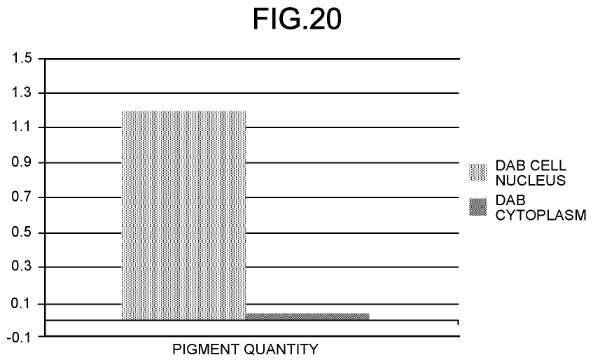

FIG. 17 is a diagram illustrating H pigment spectra of a cell nucleus and cytoplasm. FIG. 18 is a diagram illustrating H pigment quantities in the cell nucleus and cytoplasm. FIG. 19 is a diagram illustrating DAB pigment spectra of the cell nucleus and cytoplasm. FIG. 20 is a diagram illustrating DAB pigment quantities in the cell nucleus and cytoplasm. For each pixel in the input training image, the staining characteristic estimating unit 142 estimates, from the spectra of the pixels illustrated in FIG. 14 and FIG. 15, the H pigment spectra of the cell nucleus and cytoplasm illustrated in FIG. 17, the H pigment quantities in the cell nucleus and cytoplasm illustrated in FIG. 18, the DAB pigment spectra of the cell nucleus and cytoplasm illustrated in FIG. 19, and the DAB pigment quantities in the cell nucleus and cytoplasm illustrated in FIG. 20.

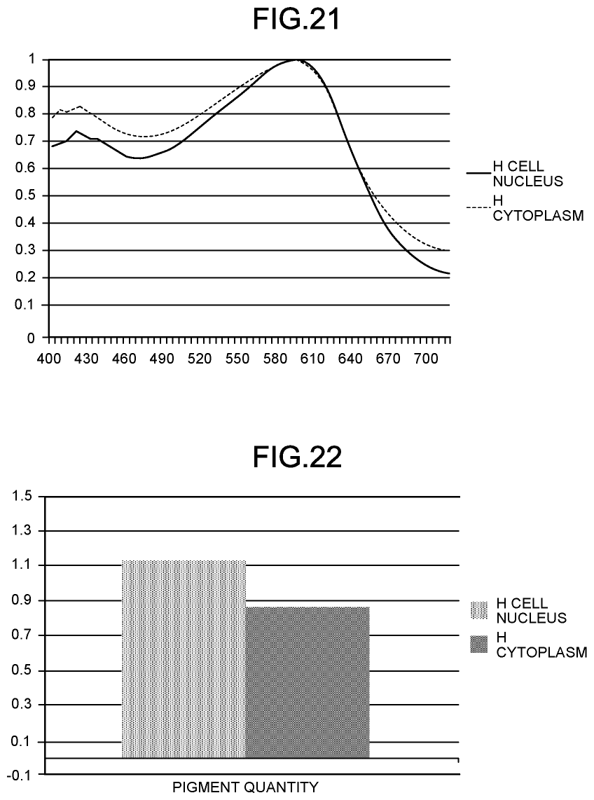

Furthermore, the staining characteristic converting unit 143 converts the staining characteristics of each tissue in the input training image, into staining characteristics of the tissue in a training image that has been recorded in the recording unit 130 (Step S16). FIG. 21 is a diagram illustrating H pigment spectra of a cell nucleus and cytoplasm in a training image for conversion. FIG. 22 is a diagram illustrating H pigment quantities of the cell nucleus and cytoplasm in the training image for conversion. FIG. 21 and FIG. 22 respectively correspond, for example, to “H pigment spectrum A1” and “H pigment quantity A1” of the protocol 1 illustrated in FIG. 13. The staining characteristic converting unit 143 then converts the H pigment spectra of the input training image illustrated in FIG. 17 into the H pigment spectra of the training image illustrated in FIG. 21 and converts the H pigment quantities in the input training image illustrated in FIG. 18 into the H pigment quantities in the training image illustrated in FIG. 22.

Based on the tissues' staining characteristics resulting from the conversion by the staining characteristic converting unit 143, the virtual image generating unit 144 repeatedly generates a virtual stained specimen image stained by a specimen preparing process protocol different from the specimen preparing process protocols of the input training image and plural training images (Step S17).

Subsequently, the arithmetic unit 140 determines whether or not conversion has been done for all tissues in all training images (Step S18). If the arithmetic unit 140 determines that conversion has not been done for all of the tissues in all of the training images, the processing is returned to Step S16 and the same processing is repeated. That is, the arithmetic unit 140 repeats the processing until conversion has been done for all of the tissues in all of the training images. As a result, conversion of the input training image for N training images prepared by different specimen preparing process protocols is done. Including both the conversion for a cell nucleus and the conversion for cytoplasm, a total of 2N virtual stained specimen images are able to be generated.

The method of calculation is similar to that of the first embodiment, but in calculation of the absorbance a′(x, y, λ), an absorbance of a cell nucleus is able to be found by Equation (7) below instead of Equation (1), and an absorbance of cytoplasm is able to be found by Equation (8) below instead of Equation (1).

$$\begin{matrix}{a}^{′} ( x , y , λ ) = \frac{{D}_{H \cdot n \cdot dest}}{{D}_{H \cdot n \cdot src}} \cdot {A}_{H \cdot n \cdot dest} ( λ ) \cdot {d}_{H} ( x , y ) + \frac{{D}_{DAB \cdot n \cdot dest}}{{D}_{DAB \cdot n \cdot src}} \cdot {A}_{DAB \cdot n \cdot dest} ( λ ) \cdot {d}_{DAB} ( x , y ) & ( 7 ) \\ {a}^{′} ( x , y , λ ) = \frac{{D}_{H \cdot c \cdot dest}}{{D}_{H \cdot c \cdot src}} \cdot {A}_{H \cdot c \cdot dest} ( λ ) \cdot {d}_{H} ( x , y ) + \frac{{D}_{DAB \cdot c \cdot dest}}{{D}_{DAB \cdot c \cdot src}} \cdot {A}_{DAB \cdot c \cdot dest} ( λ ) \cdot {d}_{DAB} ( x , y ) & ( 8 )\end{matrix}$$

As described above, according to the second embodiment, because conversion is performed with the tissue information maintained, the number of training images is able to be increased with the accuracy of diagnostic support maintained.

### Third Embodiment

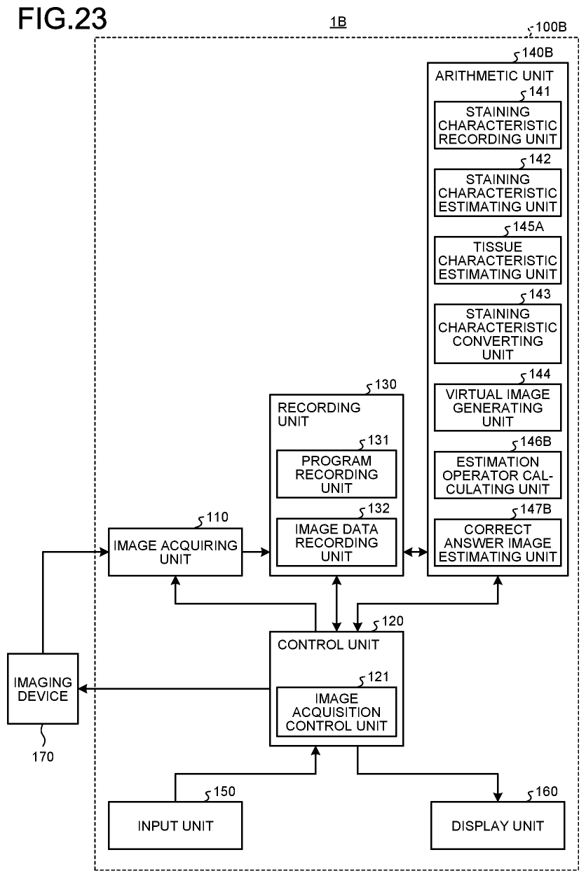

Next, a third embodiment of the disclosure will be described below. FIG. 23 is a block diagram illustrating an example of a configuration of an imaging system including an image processing apparatus according to a third embodiment of the disclosure. As illustrated in FIG. 23, an arithmetic unit 140B of an image processing apparatus 100B in an imaging system 1B includes: an estimation operator calculating unit 146B that calculates, from a data set including a specimen preparing process protocol or protocols and a correct answer image or images for plural training images or an input training image, an estimation operator for obtaining a correct answer image of an input image by estimation using regression analysis or by classification; and a correct answer image estimating unit 147B that estimates, based on the estimation operator, the correct answer image, from the input image.

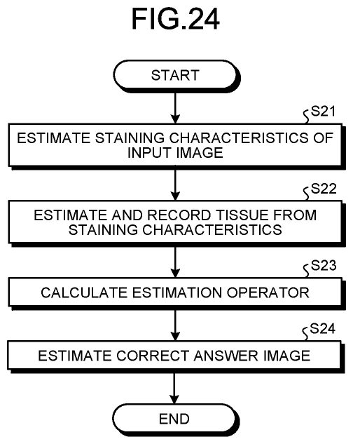

FIG. 24 is a flowchart illustrating operation of the image processing apparatus illustrated in FIG. 23. Before the processing in FIG. 24 is performed, the processing illustrated in FIG. 12 has been performed already. As illustrated in FIG. 24, a pigment spectrum and a pigment quantity that are staining characteristics of each pigment at each pixel of an input image that is a stained specimen image including staining using the plural pigments are estimated from an optical spectrum of that pixel (Step S21).

The tissue characteristic estimating unit 145A then estimates a tissue that each pixel of the input image belongs to, from the staining characteristics of the pixel, and records the specimen preparing process protocol for the input image in association with the estimated tissue, into the recording unit 130 (Step S22).

The estimation operator calculating unit 146B calculates, from a data set including a specimen preparing process protocol or protocols and a correct answer image or images for plural training images or an input training image, an estimation operator for obtaining a correct answer image for the input image by estimation using regression analysis or by classification (Step S23).

The estimation using regression analysis may involve linear regression or machine learning (including deep learning). Therefore, the estimation operator may be a regression matrix or a deep learning network.

Furthermore, the classification may involve linear discrimination or machine learning (including deep learning). Therefore, the estimation operator may be a linear discriminant function or a deep learning network.

Based on the estimation operator, the correct answer image estimating unit 147B estimates a correct answer image from the input image (Step S24).

A specific calculation method will be described below. Firstly, regression matrices may be expressed by Equations (9) and (10) below, where e, r, g, and b are pixel values of a correct answer image estimated for any coordinates on the input image, and m_{R}, m_{G}, and m_{B} are regression matrices.

$$\begin{matrix}e = [ \begin{matrix}r & g & b\end{matrix} ] [ \begin{matrix}{m}_{R} \\ {m}_{G} \\ {m}_{B}\end{matrix} ] & ( 9 ) \\ [ \begin{matrix}{e}_{1} \\ {e}_{2} \\ M \\ {e}_{N}\end{matrix} ] = [ \begin{matrix}{r}_{1} & {g}_{1} & {b}_{1} \\ {r}_{2} & {g}_{2} & {b}_{2} \\ M & M & M \\ {r}_{N} & {g}_{N} & {b}_{N}\end{matrix} ] [ \begin{matrix}{m}_{R} \\ {m}_{G} \\ {m}_{B}\end{matrix} ] & ( 10 )\end{matrix}$$

Furthermore, where E, C, and M represent matrices, they can be expressed by Equations (11) and (12).

E=CM (11)

M=(C^{T}C)⁻¹C^{T}E (12)

If a deep learning network is used, regression estimation is able to be optimized by a method described in “Image-to-Image Translation with Conditional Adversarial Networks”, arXiv:1611.07004v1 [cs.CV], 21 Nov. 2016.

If classification is performed, optimization may be done by a method described in “ImageNet Classification with Deep Convolutional Neural Networks”, Alex Krizhevsky and Sutskever, Ilya and Geoffrey E. Hinton, NIPS2012_4824.

A network that has been trained for another purpose may be utilized for transfer learning. Transfer learning facilitates estimation.

The configuration for machine learning (including deep learning) and the configuration of the correct answer image estimating unit 147B may be separately provided. Furthermore, the configuration for machine learning (including deep learning) may be provided in a server, for example, connected via an internet line.

### Modified Example 3-1

In Modified Example 3-1, a data set including immunostained images serving as training images and images serving as correct answer images in which positive cells and negative cells have been detected from immunostained images is prepared. Correct answer images may be prepared, for example, by a medical doctor manually marking regions corresponding to positive cells and negative cells in immunostained images.

Based on the data set prepared, the estimation operator calculating unit 146B calculates an estimation operator that is classification processing for classification of an input image that is an immunostained image, as a positive cell, a negative cell, or a region other than these cells.

Based on the estimation operator, the correct answer image estimating unit 147B estimates a correct answer image from the input image, the correct answer image being an image in which positive cells and negative cells have been detected. As a result, a pathologist is able to readily identify the positive cells and negative cells from the correct answer image and workload on the pathologist is thus able to be reduced.

### Modified Example 3-2

In Modified Example 3-2, a data set including HE stained images serving as training images and images serving as correct answer images in which normal regions and cancer regions have been detected from HE stained images is prepared. Correct answer images may be prepared, for example, by a medical doctor manually marking regions corresponding to normal regions and cancer regions in HE stained images.

Based on the data set prepared, the estimation operator calculating unit 146B calculates an estimation operator that is classification processing for classification of an input image that is an HE stained image, as a normal region or a cancer region.

Based on the estimation operator, the correct answer image estimating unit 147B estimates a correct answer image from the input image, the correct answer image being an image in which a normal region and a cancer region have been detected. As a result, a pathologist is able to readily identify the normal region and cancer region from the correct answer image and workload on the pathologist is thus able to be reduced.

### Modified Example 3-3

In Modified Example 3-3, a data set including multiply stained specimen images serving as training images and pigment spectrum images or pigment quantity images of each type of staining is prepared, the pigment spectrum images or pigment quantity images serving as correct answer images and being from multiply stained specimen images. Correct answer images may be calculated from spectral images.

Based on the data set prepared, the estimation operator calculating unit 146B calculates an estimation operator that is processing for estimating, from an input image that is a multiply stained specimen image, a virtual pigment spectrum image or pigment quantity image for each type of staining by regression.

Based on the estimation operator, the correct answer image estimating unit 147B estimates, from the input image, a virtual pigment spectrum image or pigment quantity image serving as a correct answer image, for each type of staining.

### Modified Example 3-4

In Modified Example 3-4, a data set including IHC stained images serving as training images and standard staining characteristic images of a standard specimen preparing process protocol is prepared, the standard staining characteristic images serving as correct answer images.

Based on the data set prepared, the estimation operator calculating unit 146B calculates an estimation operator that is processing for estimating, from an input image that is an IHC stained image, a standard staining characteristic image by regression.

Based on the estimation operator, the correct answer image estimating unit 147B estimates, from the input image, a standard staining characteristic image serving as a correct answer image.

### Modified Example 3-5

In Modified Example 3-5, a data set including tissue specimen images serving as training images and images serving as correct answer images and having tissues captured in tissue specimen images is prepared, each of the tissues having been classified as, for example, a cell nucleus or cytoplasm.

Based on the data set prepared, the estimation operator calculating unit 146B calculates an estimation operator that is classification processing for classification of an input image that is a tissue specimen image, into classes, such as a cell nucleus and cytoplasm.

Based on the estimation operator, the correct answer image estimating unit 147B estimates, from the input image, an image serving as a correct answer image and having tissues therein classified into classes, such as a cell nucleus and cytoplasm.

### Fourth Embodiment

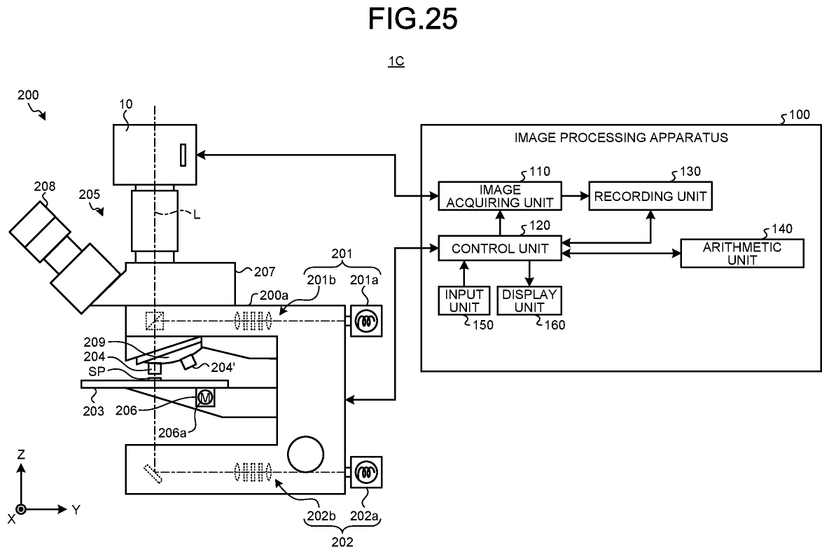

Next, a fourth embodiment of the disclosure will be described below. FIG. 25 is a block diagram illustrating an example of a configuration of an imaging system including an image processing apparatus according to the fourth embodiment of the disclosure. As illustrated in FIG. 25, an imaging system 1C according to the fourth embodiment includes: a microscope device 200 having the imaging device 170; and the image processing apparatus 100. The imaging system 1C may include, instead of the image processing apparatus 100, the image processing apparatus 100A illustrated in FIG. 11 or the image processing apparatus 100B illustrated in FIG. 23.

The microscope device 200 has: an arm 200a that includes an epi-illumination unit 201 and a transmitting illumination unit 202 and is approximately C-shaped; a specimen stage 203 that is attached to the arm 200a and is where a subject SP is placed, the subject SP being a target to be observed; an objective lens 204 provided at one end of a lens barrel 205 via a trinocular lens barrel unit 207, the objective lens 204 being opposite to the specimen stage 203; and a stage position changing unit 206 that moves the specimen stage 203. The trinocular lens barrel unit 207 branches observation light from the subject SP into: the imaging device 170 provided at the other end of the lens barrel 205; and an eyepiece unit 208 described later, the observation light being incident from the objective lens 204. The eyepiece unit 208 is for a user to directly observe the subject SP.

The epi-illumination unit 201 includes an epi-illumination light source 201a and an epi-illumination optical system 201b, and irradiates the subject SP with epi-illumination light. The epi-illumination optical system 201b includes various optical members (such as a filter unit, a shutter, a field stop, and an aperture diaphragm) that condense illumination light emitted from the epi-illumination light source 201a and guide the condensed illumination light in the direction of an observation optical path L.

The transmitting illumination unit 202 includes a transmitting illumination light source 202a and a transmitting illumination optical system 202b, and irradiates the subject SP with transmitting illumination light. The transmitting illumination optical system 202b includes various optical members (such as a filter unit, a shutter, a field stop, and an aperture diaphragm) that condense illumination light emitted from the transmitting illumination light source 202a and guide the condensed illumination light in the direction of the observation optical path L.

The objective lens 204 is attached to a revolver 209 that is able to hold plural objective lenses (for example, the objective lens 204 and an objective lens 204′) having magnifications different from one another. By rotating this revolver 209 to change the objective lens 204 or 204′ opposite to the specimen stage 203, the imaging magnification is able to be changed.

A zoom unit is provided inside the lens barrel 205, the zoom unit including: plural zoom lenses; and a drive unit that changes positions of these zoom lenses. The zoom unit magnifies or reduces a subject image within an imaging field by adjusting the position of each zoom lens.

The stage position changing unit 206 includes, for example, a drive unit 206a, such as a stepping motor, and changes the imaging field by moving the position of the specimen stage 203 in the XY plane. Furthermore, the stage position changing unit 206 matches a focal point of the objective lens 204 to the subject SP by moving the specimen stage 203 along the Z-axis.

By performing, at the imaging device 170, multiband imaging of the magnified image of the subject SP generated at the microscope device 200, a training image that is a color image of the subject SP is displayed on the display unit 160. The image processing apparatus 100, the image processing apparatus 100A, or the image processing apparatus 100B then generates a virtual stained specimen image from the training image.

The disclosure enables implementation of an operating method of an image processing apparatus, the image processing apparatus, and an operating program for the image processing apparatus that enable the number of training images to be increased while maintaining the accuracy in diagnostic support.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An operating method of an image processing apparatus, comprising:
estimating, from an optical spectrum of each pixel of each training image of plural training images, a pigment spectrum and a pigment quantity that are staining characteristics of each pigment at the pixel of the training image, the plural training images being stained specimen images prepared by plural first specimen preparing process protocols different from one another, each first specimen preparing process protocol including staining using plural pigments;
recording the first specimen preparing process protocol for the training image in association with the estimated staining characteristics;
estimating, from an optical spectrum of each pixel of an input training image, staining characteristics of each pigment at the pixel of the input training image, the input training image being a stained specimen image prepared by a second specimen preparing process protocol different from the plural first specimen preparing process protocols and including staining using the plural pigments, the input training image being input as a training image for learning;
converting the staining characteristics of at least one selected pigment in the input training image into the staining characteristics of the at least one selected pigment of any one of the plural training images; and
generating, based on the converted staining characteristics of the at least one selected pigment in the input training image, a virtual stained specimen image that is stained by a third specimen preparing process protocol different from the first specimen preparing process protocols and the second specimen preparing process protocol.

2. The operating method according to claim 1, further comprising
repeatedly performing the converting to convert the staining characteristics of each pigment in the input training image into the recorded staining characteristics of the pigment in each training image, and
repeatedly performing the generating based on the converted staining characteristics of each pigment in the input training image.

3. The operating method according to claim 1, further comprising
estimating, from the staining characteristics in each pixel of the plural training images and the input training image, a tissue to which the pixel of the plural training images or the input training image belongs,
converting the staining characteristics of at least one selected tissue in the input training image into the recorded staining characteristics of the at least one selected tissue of any one of the plural training images, and
generating, based on the converted staining characteristics in the tissue of the at least one selected tissue in the input training image, a virtual stained specimen image that is stained by a third specimen preparing process protocol different from the first specimen preparing process protocols and the second specimen preparing process protocol.

4. The operating method according to claim 1, further comprising
estimating, from an optical spectrum of each pixel of an input image that is a stained specimen image including staining using the plural pigments, staining characteristics of each pigment at the pixel of the input image,
calculating, from a data set including the first specimen preparing process protocols or the second specimen preparing process protocol and a correct answer image or images, an estimation operator for obtaining a correct answer image for the input image by estimation using regression analysis or by classification, and
estimating, based on the calculated estimation operator, a correct answer image from the input image.

5. The operating method according to claim 3, further comprising
classifying each pixel of the input training image from the staining characteristics of the pixel of the input training image,
performing classification into plural tissues according to the classification of each pixel of the input training image, and
calculating, based on the staining characteristics in pixels belonging to the classified tissues, feature data as the staining characteristics in each tissue of the classified tissues.

6. An image processing apparatus comprising a processor comprising hardware, the processor being configured to:
estimate, from an optical spectrum of each pixel of each training image of plural training images, a pigment spectrum and a pigment quantity that are staining characteristics of each pigment at the pixel of the training image, the plural training images being stained specimen images prepared by plural first specimen preparing process protocols different from one another, each first specimen preparing process protocol including staining using plural pigments;
record the first specimen preparing process protocol for the training image in association with the estimated staining characteristics;
estimate, from an optical spectrum of each pixel of an input training image, staining characteristics of each pigment at the pixel of the input training image, the input training image being a stained specimen image prepared by a second specimen preparing process protocol different from the plural first specimen preparing process protocols and including staining using the plural pigments, the input training image being input as a training image for learning;
convert the staining characteristics of at least one selected pigment in the input training image into the staining characteristics of the at least one selected pigment of any one of the plural training images; and
generate, based on the converted staining characteristics of the at least one selected pigment in the input training image, a virtual stained specimen image that is stained by a third specimen preparing process protocol different from the first specimen preparing process protocols and the second specimen preparing process protocol.

7. A non-transitory computer-readable recording medium with an executable program stored thereon, the program causing an image processing apparatus to execute:
estimating, from an optical spectrum of each pixel of each training image of plural training images, a pigment spectrum and a pigment quantity that are staining characteristics of each pigment at the pixel of the training image, the plural training images being stained specimen images prepared by plural first specimen preparing process protocols different from one another, each first specimen preparing process protocol including staining using plural pigments;
recording the first specimen preparing process protocol for the training image in association with the estimated staining characteristics;
estimating, from an optical spectrum of each pixel of an input training image, staining characteristics of each pigment at the pixel of the input training image, the input training image being a stained specimen image prepared by a second specimen preparing process protocol different from the plural first specimen preparing process protocols and including staining using the plural pigments, the input training image being input as a training image for learning;
converting the staining characteristics of at least one selected pigment in the input training image into the staining characteristics of the at least one selected pigment of any one of the plural training images; and
generating, based on the converted staining characteristics of the at least one selected pigment in the input training image, a virtual stained specimen image that is stained by a third specimen preparing process protocol different from the first specimen preparing process protocols and the second specimen preparing process protocol.
